(19) United States
(12) Patent Application Publication
(10) Pub. No.: US 2006/0246132 A1
(43) Pub. Date: Nov. 2, 2006

(54) Sustained release formulations of venlafaxine

(76) Inventor: Fjalar Johannsson, Reykjavik (IS)

Correspondence Address: WENDEROTH, LIND & PONACK, L.L.P., 2033 K STREET N. W., SUITE 800, WASHINGTON, DC, 20006-1021, US

(21) Appl. No.: 10/544,624

(22) PCT Filed: Feb. 9, 2004
(86) PCT No.: PCT/IS04/00003

§ 371 Date: Jan. 18, 2006

(30) Foreign Application Priority Data
Feb. 7, 2003 (IS) 6710
Feb. 5, 2004 (IS) 7143

(51) Int. Cl. A61K 9/22

(52) U.S. Cl. 424/468

(57) ABSTRACT

The present invention relates to sustained release tablet formulations of venlafaxine. Kollidon SR proved to be an excellent sustained release agent for venlafaxine, that is an extremely soluble drug.

### FIELD OF THE INVENTION

The present invention relates to sustained release tablet formulations of venlafaxine.

### TECHNICAL BACKGROUND AND PRIOR ART

Venlafaxine, (+/−)-[α-[(dimethylamino)methyl]-p-methoxybenzyl]cyclohexanol, is a phenylethylamine derivative which facilitates neurotransmission in the brain by blocking presynaptic reuptake of serotonin and noradrenaline for use in treating depression. See for example Holliday and Benfield, Venlafaxine, a review of its pharmacology and therapeutic potential in depression, Drugs, Vol. 49, No. 2, 1995, pp 280-294.

U.S. Pat. No. 4,535,186 describes venlafaxine and its acid additional salts.

The preparation of useful sustained release formulations of venlafaxine hydrochloride is complicated because venlafaxine HCl is very water soluble. The advantage of sustained release tablets compared to conventional tablets is that the frequency of dosage administration is reduced. Sustained release formulations can further have the advantage of inducing less side effects than conventional tablets, because the blood plasma levels of the active compound increase more slowly.

WO 9427589 concerns controlled-release dosage forms comprising venlafaxine and polymers selected from poly(alkylene oxide) polymer, cellulose polymer and maltodextrin polymer.

WO 99/22724 (EP 1028718) relates to extended release spheriod cores of venlafaxine hydrochloride. The cores are prepared by means of microcrystalline cellulose without the addition of hydroxypropylmethylcellulose. Furthermore, ethylcellulose is used as sustained release coating agent on the core in this formulation.

Sustained release tablets of venlafaxine hydrochloride are discussed in Makhija and Vavia, Once daily sustained release tablets of venlafaxine, a novel antidepressant, European Journal of Pharmaceutics and Biopharmaceutics, Vol. 54, No. 1, July 2002, pp 9-15. The article relates to matrix system based on swellable as well as non-swellable polymers. The polymers studied are hydroxypropylmethylcellulose, cellulose acetate, Eudragit RSPO and ethylcellulose.

### SUMMARY OF THE INVENTION

In an attempt to prepare a suitable sustained release tablet of venlafaxine numerous sustained release agents were tried, povidone (e.g. Kollidone), hydrogenated vegatable oil (e.g. Lubritab), polyethylene glycol (e.g. Macrogol), glyceril behenate (e.g. Compritol), polymethacrylates (e.g. Eudragit), hydroxypropylmethylcellulose (e.g. Methocel) and glyceryl palmitostearate (e.g. Precirol).

It was discovered that useful formulations of venlafaxine can be produced by use of a mixture of povidone and polyvinylacetate known as Kollidone SR.

Kollidon SR is used in various applications including preparing sustained release pharmaceutical compositions, as described in the technical and patent literature. For example, EP 0 231 826 B1 describes sustained release tablet containing theophylline as the active ingredient.

The properties of Kollidone SR are described in V. Buhler, Kollidon®, Polyvinylpyrrolidone for the pharmaceutical industry, 233-249, BASF, Ludwigshafen 2001.

Kollidone SR consists mainly of two polymers, povidone and polyvinyl acetate. The povidone part is water soluble but the polyvinyl acetate is water-insoluble. When a tablet comprising Kollidone SR is immersed in a water solution the water soluble polymer dissolves and passages are formed in the tablet. The active ingredinet will then diffuse through the passages.

Althought Kollidone SR is a known sustained release agent, the inventors were surprised to find that it was so suitable agent for venlafaxine because of the high solubility of the active material.

However, the dissolution profile of uncoated tablets that contained Kollidone SR as the only sustained-release agent showed a faster release in the beginning than was intended. In a single dose pharmacokinetic study of these tablets the Cₘₐₓ was slightly higher in the beginning than was anticipated.

In an attempt to further control the initial rate of release of venlafaxine from the tablets, several types of film materials were tested and polymethacrylates proved to be suitable and several types of this film material were extensively tested. Coating the tablets with a film that contained polymethacrylates proved to be surprisingly effective for the sustained release tablet of venlafaxine.

The polymethacrylates that were tested are mixtures of polyethyl acrylate and polymethyl methacrylate and they optionally also include trimethylammonioethyl methacrylate chloride. The trade names for the tested polymethacrylates are Eudragit RS, Eudragit RL and Eudragit NL.

By coating the tablets with a film containing Eudragit SR 30 D, the Cₘₐₓ fitted intended criteria.

Conventional hydroxypropylmethylcellulose (HPMC) based coatings do not affect the dissolution rate of sustained release tablets, since they dissolve too quikly in vivo.

Properties of polymethacrylates are described in A. H. Kibbe, Handbook of pharmaceutical excipients, 401-406, American Pharmaceutical Association, Washington, and Pharmaceutical Press, London, 2000.

Eudragit RS is a water insoluble, swellable film-former based on neutral methacrylic acid esters with a small proportion of trimethylaminoetyl methacrylate chloride. The ratio is 1:40 trimethylaminoetyl methacrylate chloride:methacrylic acid esters.

The quaternary ammonium groups determine the swellability of the films and their permeability to water, dissolved salts and medicinal substances. The small amount in the Eudragit RS result in the properties that it swells less than comparable Eudragit film formers, and is only slightly permeable to active ingredients.

### BRIEF DESCRIPTION OF FIGURES

FIG. 1 shows the effect of increasing amount of Kollidone RS on the dissolution rate of venlafaxine HCl.

FIG. 2 shows the effect of increasing the hardness of the tablets on the dissolution profile of venlafaxine HCl.

FIG. 3 shows the dissolution profiles of venlafaxine sustained release tablets in two different media, water and 0.01 M HCl. The dissolution profiles are independent of the pH.

FIG. 4 shows the dissolution profiles of uncoated tablets and tablets coated with a film containing Eudragit RS 30 D. The amount of the film on the tablet surface affects the dissolution rate.

### DETAILED DESCRIPTION

The invention provides a sustained release pharmaceutical formulation comprising pharmaceutically effective amount of venlafaxine or an acid addition salt thereof, 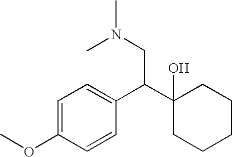
a sustained release agent selected from sustained release agent selected from povidone, a mixture of povidone and polyvinyl acetate, hydrogenated vegatable oil, polyethylene glycol, glyceril behenate and glyceril palmitostearate; and a lubricant.

The pharmaceutical formulation of the present invention comprises:

a) 15-40% w/w of venlafaxine HCl;

b) 50-85% w/w of the sustained release agent; and

c) 0.5-5.0% w/w of lubricant

and optionally a filler material and glidant.

The sustained release agent may suitably be selected from povidone (e.g. Kollidone), a mixture of povidone and polyvinyl acetate (e.g. Kollidone SR), hydrogenated vegatable oil (e.g. Lubritab), polyethylene glycol (e.g. Macrogol), glyceril behenate (e.g. Compritol), polymethacrylates (e.g. Eudragit), hydroxypropylmethylcellulese (e.g. Methocel) and glyceryl palmitostearate (e.g. Precirol).

The Kollidone SR was found to be especially suitable in controlling the release of venlafaxine. It was found that the dissolution profiles for the tablets depend on the amount of the Kollidone SR. Furthermore, it was found that the hardness of the tablets could be used to adjust the rate of the release of venlafaxine to the preferred dissolution profile. The hardness factor was especially surprising since usually the properties of Kollidone SR are not affected by the hardness of the tablets.

The lubricant is selected from magnesium stearate, hydrogenated vegatable oil, glyceryl dibehenate and sodium fumaric acid. Magnesium stearate is preferred.

For a sustained release tablet formulation containing 37.5 mg venlafaxine, the preferable amount of venlafaxine is HCl is 19-25% w/w, the preferable amount of Kollidone SR is 55-70% w/w and the preferable amount of magnesium stearate is 2-4% w/w.

For a sustained release tablet formulation containing 75 mg venlafaxine, the preferable amount of venlafaxine HCl is 19-25% w/w, the preferable amount of Kollidone SR is 55-70% w/w and the preferable amount of magnesium stearate is 2-4% w/w.

For a sustained release tablet formulation containing 150 mg venlafaxine, the preferable amount of venlafaxine HCl is 24-30% w/w, the preferable amount of Kollidone SR is 50-70% w/w and the preferable amount of magnesium stearate is 2-4% w/w.

In one embodiment the tablet is film-coated.

The film coated tablet formulation of the present invention comprises:

a) 15-40% w/w of venlafaxine;

b) 50-85% w/w of the sustained release agent;

c) 0.5-5.0% w/w of lubricant;

and optionally a filler material and/or glidant,

wherein the tablet is coated with a film wherein the film-forming material is selected from polymethacrylates.

Several film forming types of polymethacrylates were evaluated, Eudragit NE, Eudragit RL, Eudragit SR 30 D and Eudragit RS powder. Eudragit SR 30 D gave the best result.

The coating was performed by conventional pan spray coating process using solution containing the Eudragit SR 30 D (30% dispersion in water), titanium dioxide, talc, polyethylene glycol and purified water.

The time used in the coating process affects the amount of the film on the tablet surface. The amount of the film corresponding to 0.5-3.0% w/w, more preferably 1.0-2.0% w/w showed the most suitable dissolution profile for intended use as a sustained release pharmaceutical.

The coating solution includes 15-80% w/w Eudragit RS 30 D, 0.5-10% w/w titanium dioxide, 0.5-15% w/w talc, 0.5-10% w/w polyethylene glycol and 00-85% w/w purified water, preferably 30-70% w/w Eudragit RS 30 D, 1.5-6% w/w titanium dioxide, 2-8% w/w talc, 1.5-5% w/w polyethylene glycol and 25-60% w/w purified water, more preferably 45-60% w/w Eudragit RS 30 D, 2-3% w/w titanium dioxide, 3.5-5% w/w talc, 1-3% w/w polyethylene glycol and 30-50% w/w purified water and most preferably 52-54% w/w Eudragit RS 30 D, 2-3% w/w titanium dioxide, 4-5% w/w talc, 1-3% w/w polyethylene glycol and 35-43% w/w purified water.

The polyethylene glycol is preferably Macrogol 6000.

Dissolution of venlafaxine can also be adjusted by use of insoluble fillers such as calcium phosphate and microcrystalline cellulose. Calcium hydrogen phosphate dihydrate is preferred.

Optionally the formulations include glidants such as silica colloid anhydrate.

The dissolution profiles of the sustained release formulation are independent of the pH of the dissolution medium.

### EXAMPLES

The following examples are merely illustrative of the present invention and they should not be considered as limiting the scope of the invention.

### Example 1

The following materials were combined by wet granulation to produce 37.5 mg venlafaxine sustained release tablets

| | |
|---|---|
| Venlafaxine HCl | 42.5 mg |
| Kollidone SR | 127.5 mg |
| Magnesium stearate | 5 mg |

### Example 2

The following materials were combined by wet granulation to produce 75 mg venlafaxine sustained release tablets:

| | |
|---|---|
| Venlafaxine HCl | 85 mg |
| Kollidone SR | 255 mg |
| Magnesium stearate | 10 mg |

### Example 3

The following materials were combined by wet granulation to produce 150 mg venlafaxine sustained release tablets:

| | |
|---|---|
| Venlafaxine HCl | 170 mg |
| Kollidone SR | 400 mg |
| Magnesium stearate | 20 mg |

### Example 4

Dissoltion profiles of slow release tablets prepared by the inventors. Dissolution profiles for compositions that include relatively different amounts of kollidone SR.

Batch No

| | 17 | 18 | 19 | 20 |
|---|---|---|---|---|
| | Round | Round | Round | Round |
| | tablets | tablets | tablets | tablets |
| Tablets | mg | mg | mg | mg |
| Venlafaxine | 42.5 | 42.5 | 42.5 | 42.5 |
| HCl | | | | |
| Kollidone | 127.5 | 152.5 | 192.5 | 252.5 |
| Magnesium | 5.0 | 5.0 | 5.0 | 5.0 |
| stearate | | | | |
| Total | 175 | 195 | 240 | 300 |

Dissolution profiles for tablets having different amount of Kollidon SR but the same amount of venlafaxine HCl and magnesium stearate

| | Batch No. 17 | Batch No. 18 | Batch No. 19 | Batch No. 20 |
|---|---|---|---|---|
| | % dissolved | % dissolved | % dissolved | % dissolved |
| Time | mean of 6 | mean of 6 | mean of 6 | mean of 6 |
| min | tablets | tablets | tablets | tablets |
| 0 | 0.3 | 0.0 | 0.0 | 0.0 |
| 30 | 24.1 | 24.6 | 20.5 | 16.2 |
| 60 | 32.5 | 32.9 | 27.5 | 22.5 |
| 120 | 42.1 | 42.0 | 35.2 | 29.4 |
| 180 | 48.5 | 47.8 | 40.2 | 33.8 |
| 240 | 53.5 | 52.6 | 44.0 | 37.3 |
| 300 | 58.0 | 56.9 | 47.1 | 40.0 |
| 360 | 62.1 | 60.7 | 50.0 | 42.5 |
| 420 | 65.8 | 64.1 | 52.9 | 44.9 |
| 480 | 69.0 | 67.2 | 55.4 | 47.1 |
| 540 | 72.1 | 70.0 | 57.8 | 49.2 |
| 600 | 74.6 | 72.6 | 60.2 | 51.1 |
| 660 | 76.9 | 74.8 | 62.2 | 53.1 |
| 720 | 79.0 | 76.8 | 64.2 | 55.1 |
| 780 | 80.9 | 78.5 | 66.2 | 57.0 |
| 840 | 82.4 | 80.2 | 68.0 | 58.7 |
| 900 | 84.1 | 81.7 | 69.6 | 60.5 |
| 960 | 85.2 | 82.9 | 71.3 | 61.9 |
| 1020 | 86.5 | 84.1 | 72.7 | 63.3 |
| 1080 | 87.5 | 85.2 | 74.0 | 64.7 |
| 1140 | 88.3 | 86.1 | 75.3 | 65.8 |
| 1200 | 89.0 | 86.9 | 76.6 | 67.0 |
| 1260 | 89.7 | 87.6 | 77.7 | 68.0 |
| 1320 | 90.3 | 88.4 | 78.9 | 69.0 |
| 1380 | 90.7 | 89.0 | 79.8 | 69.9 |
| 1440 | 91.1 | 89.5 | 80.8 | 70.9 |

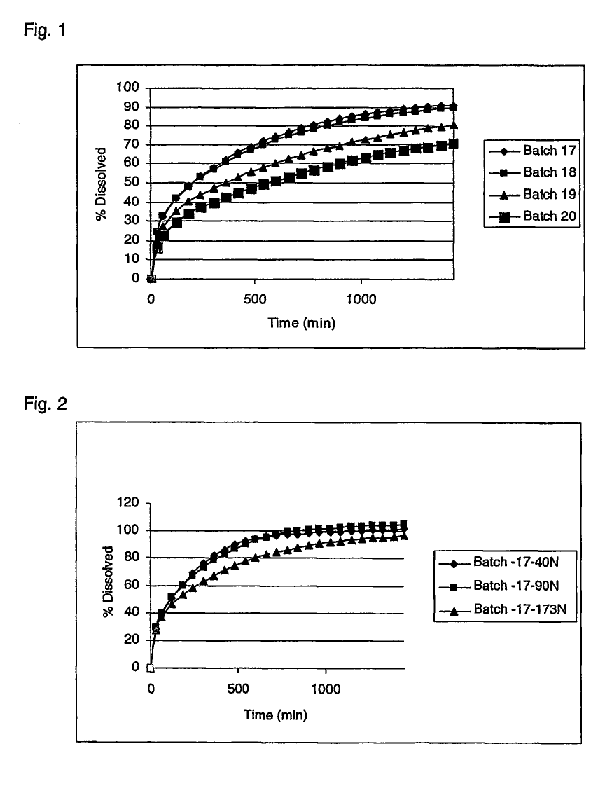

FIG. 1 shows the effect of increasing amount of Kollidone SR on the olution rate of venlafaxine HCl

### Example 5

Dissolution profiles for tablets that include the same amount of Kollidone SR but the hardness of the tablets is different.

| | Batch No. 17 | Batch No. 17 | Batch No. 17 |
|---|---|---|---|
| | Hardness 40N | Hardness 90N | Hardness 9 173N |
| Time | % dissolved | % dissolved | % dissolved |
| min | mean of 6 tablets | mean of 6 tablets | mean of 6 tablets |
| 0 | 0 | 0 | 0 |
| 30 | 27.8 | 29.3 | 27.1 |
| 60 | 38 | 39.6 | 36.2 |
| 120 | 50.6 | 51.4 | 46.3 |
| 180 | 60.7 | 59.8 | 53 |
| 240 | 69 | 66.8 | 58.3 |
| 300 | 75.9 | 73 | 63 |
| 360 | 81.8 | 78.5 | 67.3 |
| 420 | 86.4 | 83.1 | 71.1 |
| 480 | 89.9 | 87.1 | 74.4 |
| 540 | 92.5 | 90.5 | 77.4 |
| 600 | 94.4 | 93.2 | 80.1 |
| 660 | 95.6 | 95.5 | 82.4 |
| 720 | 96.7 | 97.4 | 84.5 |
| 780 | 97.5 | 98.9 | 86.4 |
| 840 | 98 | 100 | 87.9 |
| 900 | 98.4 | 101 | 89.4 |
| 960 | 98.9 | 101.6 | 90.7 |
| 1020 | 99.3 | 102.2 | 91.9 |
| 1080 | 99.6 | 102.8 | 92.9 |
| 1140 | 100 | 103.1 | 93.7 |
| 1200 | 100.2 | 103.5 | 94.4 |
| 1260 | 100.6 | 103.9 | 95 |
| 1320 | 100.9 | 104.2 | 95.5 |
| 1380 | 101.1 | 104.5 | 96.1 |
| 1440 | 101.4 | 104.7 | 96.5 |

FIG. 2 shows the effect of increasing the hardness of the tablets on the dissolution profile of venlafaxine 37.5 mg sustained release tablets

### Example 6

Dissolution profiles of Venlafaxine sustained release tablets (same batch) are independent of the dissolution medium as shown in following table.

| | % Dissolved | % Dissolved |
|---|---|---|
| | Batch 14-0.01NHCl | Batch-14-water |
| Hours | Mean of 6 tablets | Mean of 6 tablets |
| 0 | 0.0 | 0.0 |
| 1 | 27.2 | 27.5 |
| 2 | 36.8 | 37.2 |
| 3 | 43.5 | 43.8 |
| 4 | 48.7 | 48.9 |
| 5 | 53.1 | 53.3 |
| 6 | 57.1 | 57.2 |
| 7 | 60.8 | 60.8 |
| 8 | 64.2 | 64.2 |
| 9 | 67.3 | 67.3 |
| 10 | 70.3 | 70.2 |
| 11 | 73.0 | 72.9 |
| 12 | 75.5 | 75.3 |
| 13 | 77.8 | 77.6 |
| 14 | 79.9 | 79.8 |
| 15 | 81.9 | 81.8 |
| 20 | 89.8 | 89.7 |
| 24 | 94.3 | 94.2 |

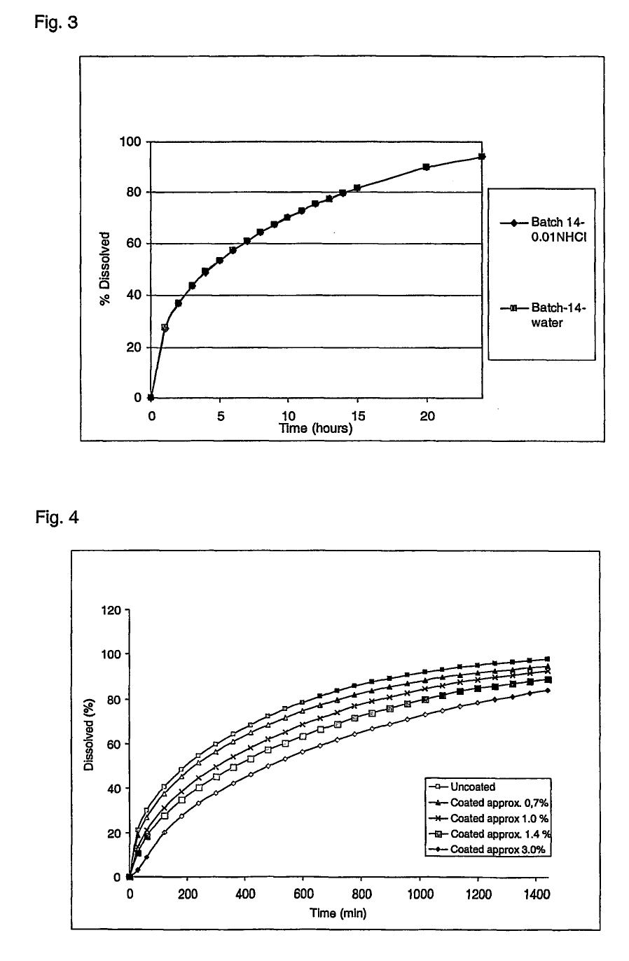

FIG. 3 showes the dissolution profiles.

### Example 7

75 mg sustained release venlafaxine tablets were prepared by combining the following materials by wet granulation:

| | | |
|---|---|---|
| Venlafaxine HCl | 22% | w/w |
| Kollidone SR | 66.3% | w/w |
| Magnesium stearate | 2.6% | w/w |
| Calcium hydrogen phosphate dihydrate | 8.3% | w/w |
| Silica colloid anhydrate | 0.8% | w/w |

The tablets were coated with Eudragit RS 30 D for different periods of time, resulting in 0.7% w/w film/tablet, 1.0% w/w film/tablet, 1.4% w/w film/tablet and 3.0% w/w film/tablet or not coated at all, for comparison in a dissolution test.

The coating liquid includes:

| | | |
|---|---|---|
| Eudragit RS 30 D | 53.00% | w/w |
| Titanium dioxide | 2.21% | w/w |
| Talc | 4.42% | w/w |
| Macrogol 6000 | 1.90% | w/w |
| Purified water | 38.47% | w/w |

Dissolution profiles of uncoated tablets and tablets with different amount of coating.

| | | Coating | Coating | Coating | Coating |
|---|---|---|---|---|---|
| | | 0.7% | 1.0% | 1.4% | 3.0% |
| Time | Uncoated % | w/w % | w/w % | w/w % | w/w % |
| [min] | dissolved | dissolved | dissolved | dissolved | dissolved |
| 0 | 0.0 | 0.0 | 0.0 | 0.0 | 0.0 |
| 30 | 21.0 | 18.7 | 13.4 | 10.7 | 3.1 |
| 60 | 29.9 | 26.9 | 21.1 | 18.5 | 8.9 |
| 120 | 40.7 | 37.7 | 31.2 | 28.0 | 20.2 |
| 180 | 48.5 | 45.3 | 38.7 | 34.9 | 27.7 |
| 240 | 54.5 | 51.3 | 44.7 | 40.5 | 33.3 |
| 300 | 59.8 | 56.4 | 49.7 | 45.3 | 38.0 |
| 360 | 64.3 | 60.8 | 54.2 | 49.6 | 42.2 |
| 420 | 68.4 | 64.8 | 58.2 | 53.4 | 46.2 |
| 480 | 72.1 | 68.3 | 61.9 | 57.1 | 49.8 |
| 540 | 75.5 | 71.6 | 65.3 | 60.4 | 53.1 |
| 600 | 78.5 | 74.6 | 68.5 | 63.5 | 56.3 |
| 660 | 81.2 | 77.2 | 71.4 | 66.4 | 59.2 |
| 720 | 83.6 | 79.6 | 74.1 | 69.0 | 61.9 |
| 780 | 85.7 | 81.8 | 76.7 | 71.5 | 64.4 |
| 840 | 87.5 | 83.7 | 78.9 | 73.8 | 66.8 |
| 900 | 89.2 | 85.4 | 80.9 | 75.9 | 69.0 |
| 960 | 90.6 | 87.0 | 82.8 | 78.0 | 71.2 |
| 1020 | 91.9 | 88.3 | 84.5 | 79.9 | 73.1 |
| 1080 | 93.0 | 89.6 | 86.0 | 81.8 | 74.9 |
| 1140 | 94.4 | 90.6 | 87.4 | 83.5 | 76.7 |
| 1200 | 95.0 | 91.6 | 88.6 | 84.9 | 78.3 |
| 1260 | 95.8 | 92.5 | 89.7 | 85.6 | 79.8 |
| 1320 | 96.5 | 93.2 | 90.7 | 86.8 | 81.2 |
| 1380 | 97.1 | 93.9 | 91.6 | 87.9 | 82.5 |
| 1440 | 97.6 | 94.5 | 92.5 | 88.9 | 83.8 |

FIG. 4 shows the dissolution profiles.

Examples 1-3 show typical compositions of venlafaxine HCl, Kollidone SR and magnesium stearate.

Example 4 shows different dissolution profiles for various concentrations of Kollidon SR.

Example 5 shows different dissolution profiles for identical compositions with various hardness of tablets.

Example 6 shows that the dissolution profiles are independent of the pH.

Example 7 shows the dissolution profiles of coated and uncoated tablets.

By using Kollidone SR it was possible to adjust the dissolution profile of venlafaxine to a satisfactory level by changing the amount of Kollidone SR and the hardness of the tablets. By further employing Eudragit RS 30 D as a coating agent the Cₘₐₓ fits the intended critera.

1. A sustained release tablet formulation comprising:
a) pharmaceutical effective amount of venlafaxine or an acid addition salt thereof;
b) a sustained release agent selected from povidone, a mixture of povidone and polyvinyl acetate, hydrogenated vegatable oil, polyethylene glycol, glyceryl behenate and glyceryl palmitostearate; and
c) a lubricant,
optionally in combination with a filling material and/or other excipients.

2. The sustained release tablet formulation of claim 1, comprising:
a) 15-30% w/w of venlafaxine HCl;
b) 50-85% w/w of a sustained release agent selected from povidone, a mixture of povidone and polyvinyl acetate, hydrogenated vegatable oil, polyethylene glycol, glyceryl behenate and glyceryl palmitostearate; and
c) 0.5-5.0% w/w of a lubricant,
optionally in combination with a filling material and/or other excipients.

3. The sustained release tablet formulation of claim 1, wherein the sustained release agent is a mixture of povidone and polyvinyl acetate.

4. The sustained release tablet formulation of claim 3, wherein the sustained release agent is Kollidone SR.

5. The sustained release tablet formulation of claim 1, wherein the lubricant is selected from magnesium stearate, hydrogenated vegetable oil, glyceryl dibehenate and sodium fumaric acid.

6. The sustained release tablet formulation of claim 4, wherein the lubricant is magnesium stearate.

7. The formulation of claim 1, which comprises venlafaxine HCl, Kollidone SR and magnesium stearate.

8. The sustained release tablet formulation of claim 1 containing 37.5 mg venlafaxine, wherein the amount of venlafaxine HCl is 19-25% w/w, the amount of Kollidone SR is 55-70% w/w and the amount of magnesium stearate is 2-4% w/w.

9. The sustained release tablet formulation of claim 1 containing 75 mg venlafaxine, wherein the amount of venlafaxine HCl is 19-25% w/w, the amount of Kollidone SR is 55-70% w/w and the amount of magnesium stearate is 2-4% w/w.

10. The sustained release tablet formulation of claim 1 containing 150 mg venlafaxine, wherein the amount of venlafaxine HCl is 24-30% w/w, the amount of Kollidone SR is 50-70% w/w and the amount of magnesium stearate is 2-4% w/w.

11. The sustained release tablet formulation of claim 1 additionally including filling material selected from calcium phosphate and microcrystalline cellulose.

12. The sustained release tablet formulation of claim 11 wherein the filling material is calcium hydrogen phosphate dihydrate.

13. The sustained release tablet formulation of claim 1 additionally including silica colloid anhydrate.

14. The sustained release tablet formulation of claim 1 wherein the tablet is film coated.

15. The sustained release tablet formulation of claim 14, wherein the film coating comprises polymethacrylate.

16. The sustained release tablet formulation of claim 15, wherein the polymethacrylate is selected from Eudragit RS, Eudragit RL and Eudragit NE.

17. The sustained release tablet formulation of claim 16, wherein the polymethacrylate is Eudragit RS.

18. The sustained release tablet formulation of claim 17, wherein the polymethacrylate is Eudragit RS 30 D.

19. The film coated sustained release tablet formulation of claim 14, wherein the coating solution includes 15-80% w/w Eudragit RS 30 D, 0.5-10% w/w titanium dioxide, 0.5-15% w/w talc, 0.5-10% w/w polyethylene glycol and 20-85% w/w purified water.

20. The film coated sustained release tablet formulation in claim 19, wherein the coating solution includes 30-70% w/w Eudragit RS 30 D, 1.5-6% w/w titanium dioxide, 2-8% w/w talc, 1.5-5% w/w polyethylene glycol and 25-60% w/w purified water.

21. The film coated sustained release tablet formulation of claim 20 wherein the coating solution includes 45-60% w/w Eudragit RS 30 D, 2-3% w/w titanium dioxide, 3.5-5% w/w talc, 1-3% w/w polyethylene glycol and 30-50% w/w purified water.

22. The film coated sustained release tablet formulation of claim 21 wherein the coating solution includes 52-54% w/w Eudragit RS 30 D, 2-3% 25 w/w titanium dioxide, 4-5% w/w talc, 1-3% w/w polyethylene glycol and 35-43% w/w purified water.

23. The film coated sustained release tablet formulation of claim 19, wherein the polyethylene glycol is Macrogol 6000.

24. The film coated sustained release tablet formulation of claim 14, wherein the amount of the film on the tablet is 0.5-3.0% w/w.

25. The film coated sustained release formulation of claim 24, wherein the amount of the film on the tablet is 1.0-2.0% w/w.
